(19) United States
(12) Patent Application Publication
(10) Pub. No.: US 2026/0061118 A1
(43) Pub. Date: Mar. 5, 2026

(54) BASAL BOLUS TREATMENT DEVICES AND METHODS

(71) Applicant: QUASURAS, INC., San Diego, CA (US)

(72) Inventors: Paul M. DIPERNA, Escondido, CA (US); James BESSER, San Juan, PR (US); Stephen DALY, Henderson, NV (US); David MAGGS, Reno, NV (US)

(21) Appl. No.: 19/309,936

(22) Filed: Aug. 26, 2025

Related U.S. Application Data
(60) Provisional application No. 63/687,697, filed on Aug. 27, 2024.

(51) Int. Cl. A61M 5/142

(52) U.S. Cl. CPC A61M 5/142; A61M 2005/14208; A61M 2205/3303; A61M 2205/502; A61M 2230/005; A61M 2230/201

(57) ABSTRACT

A drug delivery system for administering a GLP-1 agonist therapy includes a reservoir configured to contain a GLP-1 agonist, a pump in fluid communication with the reservoir and configured to deliver the GLP-1 agonist, a controller operatively coupled to the pump, and a user interface operatively coupled to the controller. The controller is programmed to deliver a basal dose of the GLP-1 agonist continuously over a 24-hour period and deliver at least one bolus dose of the GLP-1 agonist in addition to the basal dose. The system allows for independent adjustment of basal and bolus doses based on patient-specific parameters, enabling personalized GLP-1 agonist therapy administration.

### RELATED APPLICATION

The present application claims priority to U.S. Provisional Patent Application No. 63/687,697 filed on Aug. 27, 2024 and titled “Basal Bolus Treatment Devices and Methods” the entirety of which is incorporated by reference herein.

### FIELD OF INVENTION

The present disclosure relates to drug delivery systems and methods, and more particularly to devices and methods for administering GLP-1 agonist therapies using programmable basal and bolus dosing regimens.

### BACKGROUND

Diabetes management has long been a focus of medical research and treatment, with various approaches developed to help patients control their blood glucose levels. One class of medications that has gained prominence in recent years is GLP-1 (glucagon-like peptide-1) agonists. These drugs mimic the effects of the naturally occurring GLP-1 hormone, which plays a role in regulating blood sugar, appetite, and digestion.

Traditional GLP-1 agonist therapies have typically been administered as once-weekly injections, providing a standardized dose to patients. This approach has been adopted due to its convenience for patients and potential to improve adherence to treatment regimens. The effects of these medications can include improved glycemic control, weight loss, and potential benefits for cardiovascular health.

However, the administration of GLP-1 agonists as fixed weekly doses may not always provide optimal results for all patients. Some individuals may experience variable efficacy throughout the dosing period, with the effects potentially diminishing as they approach the end of the weekly cycle. Additionally, gastrointestinal side effects are common with GLP-1 agonist therapies and can impact patient tolerance and adherence to treatment.

The concept of basal-bolus insulin therapy, which involves a combination of long-acting (basal) and short-acting (bolus) insulin doses, has been well-established in diabetes management. This approach allows for more precise control of blood glucose levels by mimicking the body's natural insulin production patterns. However, the application of similar principles to GLP-1 agonist therapies has not been widely explored.

As research in diabetes management continues to evolve, there is ongoing interest in developing more personalized and flexible treatment options. This includes exploring new delivery methods and dosing regimens that could potentially enhance the efficacy of GLP-1 agonist therapies while minimizing side effects and improving patient outcomes.

### SUMMARY

In one aspect, a drug delivery system for administering a GLP-1 agonist therapy includes: a reservoir configured to contain a GLP-1 agonist; a pump in fluid communication with the reservoir and configured to deliver a GLP-1 agonist; a controller operatively coupled to the pump; and a user interface operatively coupled to the controller, wherein the controller is programmed to: deliver a basal dose of the GLP-1 agonist continuously over a 24-hour period; and deliver at least one bolus dose of the GLP-1 agonist in addition to the basal dose.

Additionally or alternatively, the basal dose and the at least one bolus dose are independently adjustable.

Additionally or alternatively, the controller is further programmed to adjust the basal dose and the at least one bolus dose based on patient-specific parameters.

Additionally or alternatively, the patient-specific parameters include at least one of body weight, meal size, time of day, or glucose levels.

Additionally or alternatively, the controller is programmed to deliver multiple bolus doses at different times within a 24-hour period.

Additionally or alternatively, the basal dose comprises 10% to 90% of a total daily dose of the GLP-1 agonist.

Additionally or alternatively, the drug delivery system further comprises a reservoir containing the GLP-1 agonist, wherein the GLP-1 agonist is a short-acting GLP-1 receptor agonist.

Additionally or alternatively, the user interface is configured to receive user input for adjusting the basal dose and the at least one bolus dose.

Additionally or alternatively, the drug delivery system further comprises a glucose sensor operatively coupled to the controller, wherein the controller is programmed to adjust the basal dose and the at least one bolus dose based on glucose readings from the glucose sensor.

Additionally or alternatively, the controller is programmed to deliver the at least one bolus dose in response to a meal-related input received through the user interface.

In another aspect, a method of administering a GLP-1 agonist therapy includes: delivering a basal dose of a GLP-1 agonist continuously over a 24-hour period using a pump; and delivering at least one bolus dose of the GLP-1 agonist in addition to the basal dose using the pump.

Additionally or alternatively, the method further comprises independently adjusting the basal dose and the at least one bolus dose.

Additionally or alternatively, the method further comprises adjusting the basal dose and the at least one bolus dose based on patient-specific parameters.

Additionally or alternatively, the patient-specific parameters include at least one of body weight, meal size, time of day, or glucose levels.

Additionally or alternatively, delivering the at least one bolus dose comprises delivering multiple bolus doses at different times within the 24-hour period.

Additionally or alternatively, the basal dose comprises 10% to 90% of a total daily dose of the GLP-1 agonist.

Additionally or alternatively, the GLP-1 agonist is a short-acting GLP-1 receptor agonist.

Additionally or alternatively, the method further comprises receiving user input through a user interface for adjusting the basal dose and the at least one bolus dose.

Additionally or alternatively, the method further comprises adjusting the basal dose and the at least one bolus dose based on glucose readings from a glucose sensor operatively coupled to the pump.

Additionally or alternatively, delivering the at least one bolus dose comprises delivering the bolus dose in response to a meal-related input received through a user interface.

### BRIEF DESCRIPTION OF FIGURES

The above and further advantages of this invention may be better understood by referring to the following description in conjunction with the accompanying drawings, in which like numerals indicate like structural elements and features in the various figures. For clarity, not every element may be labeled in every figure. The drawings are not necessarily to scale, emphasis instead being placed upon illustrating the principles of the invention.

FIG. 1 illustrates a system diagram of a drug delivery system, according to aspects of the present disclosure.

FIG. 2 depicts a system diagram of a controller for managing insulin dosing, according to an embodiment.

FIG. 3 shows a flowchart for a method of administering a GLP-1 agonist therapy, in accordance with example embodiments.

FIG. 4 illustrates a flowchart for adjusting GLP-1 agonist therapy based on patient-specific parameters, according to an aspect of the present disclosure.

FIG. 5 depicts a flowchart for administering GLP-1 agonist therapy with multiple bolus doses, according to aspects of the present disclosure.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

In brief overview, the present disclosure provides a drug delivery system for administering GLP-1 agonist therapy. The system allows for flexible and variable delivery of both basal and bolus dosing while meeting safety bounds of overall chronic exposure. The drug delivery system enables patient-friendly initiation of dosing, such as up-titration of basal dosing followed by addition of bolus dosing later. The system also allows for customization of the overall balance between basal and bolus exposure for individual patients. Additionally, the drug delivery system provides for delivery of variable bolus doses of a short-acting GLP-1 agonist at mealtimes, with the ability to adjust bolus size according to patient tolerability and meal size requirements.

Thus, embodiments described herein provide for an alternative to a QW (once weekly) GLP1 therapy, where instead the continuous delivery of a short acting GLP1 from an external pump can be programmed for variable (gradual or rapid) titration per tolerability (for improved persistence) until the personalized target basal dose is attained.

The pump platform as contemplated herein may allow for flexibility and variability in delivering both basal and bolus dosing while meeting the safety bounds of overall chronic exposure.

The pump platform may further allow for patient-friendly initiation of dosing (example: up-titration of basal and then add bolus later).

Still further, the pump platform may allow for an overall balance of basal vs bolus exposure that can be customized or individualized for the patient (example: 40% basal vs 60% basal).

The pump platform may allow for delivery of a variable bolus of the short acting GLP1 at mealtime. Like the basal rate, the bolus size can be adjusted according to patient tolerability until the personalized per meal target dose is attained. Additionally, the bolus dosing can be customized through the day by meal size requirements.

Moreover, the pump platform may allow for patient-friendly cessation or reduction of dosing if GI side effects are prohibitive or if life circumstance dictates. Use of an external pump will readily allow for hourly or daily drug holidays (temporary suspension of delivery and/or indefinite removal of pump).

This approach may be also applicable to GLP1-based multi-agonist therapies either in combination (a combination liquid in a single chamber) or multiple agents in a multiple chamber pump platform.

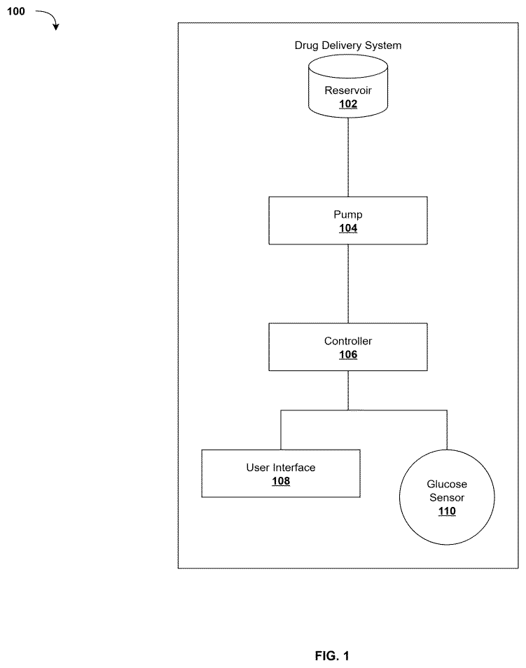

FIG. 1 illustrates a system diagram of a drug delivery system 100. The drug delivery system 100 includes components for automated medication administration based on physiological feedback.

The drug delivery system 100 comprises a reservoir 102 configured to contain a GLP-1 agonist. In some cases, the reservoir 102 may contain a short-acting GLP-1 receptor agonist. The drug delivery system 100 further includes a pump 104 in fluid communication with the reservoir 102. The pump 104 may be configured to deliver the GLP-1 agonist from the reservoir 102.

A controller 106 may be operatively coupled to the pump 104. The controller 106 may manage the overall operation of the drug delivery system 100, including controlling the delivery of the GLP-1 agonist through the pump 104.

The drug delivery system 100 also includes a user interface 108 operatively coupled to the controller 106. The user interface 108 may allow for user interaction and system configuration, enabling patients or healthcare providers to input parameters or adjust settings for GLP-1 agonist delivery.

In some cases, the drug delivery system 100 further comprises a glucose sensor 110 operatively coupled to the controller 106. The glucose sensor 110 may monitor glucose levels and provide data to the controller 106, allowing for adjustment of GLP-1 agonist delivery based on real-time glucose readings.

The components of the drug delivery system 100 are interconnected to enable coordinated functionality. The reservoir 102 may be connected to the pump 104, allowing for a direct flow path of the GLP-1 agonist. The pump 104 may be linked to the controller 106, enabling regulated medication dispensing. The controller 106 may be connected to both the user interface 108 and the glucose sensor 110, facilitating data exchange and system control.

In operation, the drug delivery system 100 may utilize feedback from the glucose sensor 110 to inform medication administration. The controller 106 may process sensor data and user inputs from the interface 108 to determine appropriate dosing. The pump 104 may then draw the GLP-1 agonist from the reservoir 102 and deliver it based on instructions from the controller 106.

The drug delivery system 100 may allow for hourly or daily drug holidays through temporary suspension of delivery or indefinite removal of the pump 104. This feature provides flexibility in treatment regimens and may improve patient compliance.

In some cases, the drug delivery system 100 may be applicable to GLP1-based multi-agonist therapies. These therapies may be administered either as a combination liquid in a single chamber of the reservoir 102 or as multiple agents in a multiple chamber pump platform.

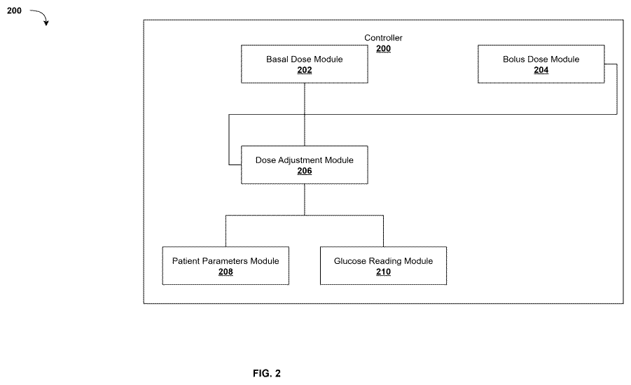

FIG. 2 illustrates a system diagram of a controller 200 for managing GLP-1 agonist dosing. The controller 200 comprises several interconnected modules that work together to determine and adjust appropriate dosing regimens.

The controller 200 includes a basal dose module 202 and a bolus dose module 204. The basal dose module 202 may be programmed to manage the continuous delivery of a basal dose of the GLP-1 agonist over a 24-hour period. The bolus dose module 204 may be configured to manage the delivery of at least one bolus dose of the GLP-1 agonist in addition to the basal dose.

A dose adjustment module 206 may be operatively connected to both the basal dose module 202 and the bolus dose module 204. The dose adjustment module 206 may allow for independent adjustment of the basal dose and the bolus dose(s). This independent adjustability may enable customization of the overall balance between basal and bolus exposure for individual patients.

The controller 200 further includes a patient parameters module 208. The patient parameters module 208 may store and process patient-specific information such as body weight, meal size, and time of day. This module may provide data to the dose adjustment module 206 to facilitate personalized dose adjustments.

A glucose reading module 210 may also be incorporated into the controller 200. The glucose reading module 210 may receive and process glucose readings from the glucose sensor 110 of the drug delivery system 100. These glucose readings may be used by the dose adjustment module 206 to further refine the basal and bolus doses.

In operation, the controller 200 may be programmed to allow for variable titration of the basal dose. This titration may be gradual or rapid, depending on patient tolerability, until a personalized target basal dose is attained. The controller 200 may also be configured to deliver multiple bolus doses at different times within a 24-hour period.

The controller 200 may be programmed to receive user input through the user interface 108 for adjusting the basal dose and the bolus dose(s). In some cases, the controller 200 may deliver a bolus dose in response to a meal-related input received through the user interface 108. The controller 200 may allow for customization of bolus dosing throughout the day based on meal size requirements.

The dose adjustment module 206 may be programmed to adjust doses based on various factors. These factors may include patient-specific parameters from the patient parameters module 208 and glucose readings from the glucose reading module 210. This multi-faceted approach to dose adjustment may allow for flexibility and variability in delivering both basal and bolus dosing while meeting safety bounds of overall chronic exposure.

In some cases, the controller 200 may be programmed to allow for patient-friendly initiation of dosing. For example, the controller 200 may implement an up-titration of the basal dose before adding bolus doses later in the treatment regimen.

The controller 200 may also be configured to allow for patient-friendly cessation or reduction of dosing. This feature may be utilized if gastrointestinal side effects become prohibitive or if life circumstances dictate a need for temporary dose reduction. The modular architecture of the controller 200 allows for such flexible management of GLP-1 agonist therapy, potentially improving patient compliance and treatment outcomes.

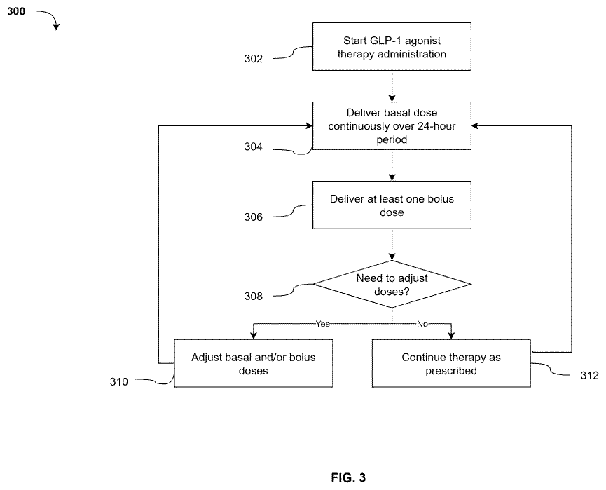

FIG. 3 illustrates a flowchart for a method of administering a GLP-1 agonist therapy using the drug delivery system 100. The method begins with a step 300 of initiating GLP-1 agonist therapy administration.

Following initiation, the method proceeds to a step 302 of delivering a basal dose of the GLP-1 agonist continuously over a 24-hour period using the pump 104. In some cases, the basal dose may comprise 10% to 90% of a total daily dose of the GLP-1 agonist. The total daily dosing exposure for the GLP-1 agonist therapy may range from 20% to 200% of the total daily recommended dose by US FDA labeling.

The method then moves to a step 304 of delivering at least one bolus dose of the GLP-1 agonist in addition to the basal dose using the pump 104. In some cases, the number of discrete bolus doses may range from one to ten within a 24-hour period. The bolus doses may be varied at each time point across the 24-hour period.

After delivering the basal and bolus doses, the method proceeds to a decision point 306 where a determination is made whether there is a need to adjust doses. This determination may be based on various factors such as glucose readings from the glucose sensor 110, patient-specific parameters from the patient parameters module 208, or user input received through the user interface 108.

If adjustment is needed, the method follows the “Yes” branch to a step 310 where the basal and/or bolus doses are adjusted. The dose adjustment module 206 may perform these adjustments based on the determined need. The total daily dose, dose balance between basal and net bolus, and bolus dosing at discrete time points may be varied over time per clinical need.

If no adjustment is needed, the method follows the “No” branch to a step 312 where the therapy is continued as prescribed.

After either adjusting the doses or continuing the therapy as prescribed, the method may loop back to step 304 to continue delivering the basal dose for the next 24-hour period. This cyclical process allows for ongoing administration and adjustment of the GLP-1 agonist therapy based on patient needs and clinical requirements.

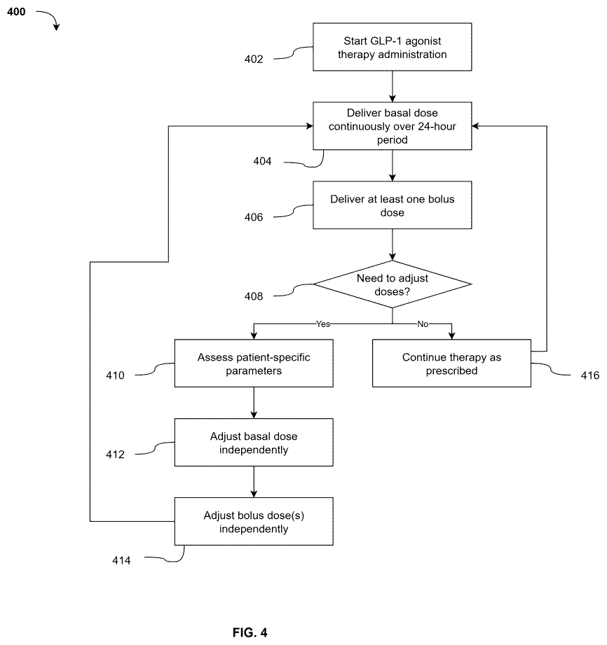

FIG. 4 illustrates a flowchart for a method of administering and adjusting GLP-1 agonist therapy based on patient-specific parameters using the drug delivery system 100. The method begins with a step 402 of initiating GLP-1 agonist therapy administration.

Following initiation, the method proceeds to a step 404 of delivering a basal dose of the GLP-1 agonist continuously over a 24-hour period using the pump 104. The method then moves to a step 406 of delivering at least one bolus dose of the GLP-1 agonist in addition to the basal dose using the pump 104.

After delivering the basal and bolus doses, the method proceeds to a decision point 408 where a determination is made whether there is a need to adjust doses. This determination may be based on various factors, including patient-specific parameters.

If adjustment is needed, the method follows the “Yes” branch to a step 410 where patient-specific parameters are assessed. In some cases, these patient-specific parameters may include body weight, meal size, time of day, or glucose levels. The patient parameters module 208 of the controller 200 may store and process this patient-specific information.

Following the assessment of patient-specific parameters, the method proceeds to a step 412 where the basal dose is adjusted independently. The basal dose module 202 and the dose adjustment module 206 of the controller 200 may work together to perform this adjustment.

The method then moves to a step 414 where the bolus dose(s) are adjusted independently. The bolus dose module 204 and the dose adjustment module 206 may collaborate to execute this adjustment.

In some cases, the method may include receiving user input through the user interface 108 for adjusting the basal dose and the at least one bolus dose. This user input may be processed by the controller 200 to further refine the dose adjustments.

The method may also include adjusting the basal dose and the at least one bolus dose based on glucose readings from the glucose sensor 110 operatively coupled to the pump 104. The glucose reading module 210 of the controller 200 may receive and process these glucose readings, which may then be used by the dose adjustment module 206 to further refine the basal and bolus doses.

After these adjustments, the method loops back to step 404 to continue the therapy with the new dosages. If no adjustment is needed at decision point 408, the method moves to a step 416 where therapy is continued as prescribed.

In some cases, the method may allow for different approaches to up-titration of dosing at initiation. These approaches may include introduction of basal dosing first, introduction of bolus dosing first, or introduction of both simultaneously. The controller 200 may be programmed to implement these different up-titration strategies based on patient needs and clinical requirements.

The method may also provide for various approaches to reduction or cessation of treatment. These approaches may include complete cessation, halting or reducing bolus doses first, halting or reducing basal dosing first, or reducing both components in tandem. The controller 200 may be configured to execute these different reduction or cessation strategies as needed.

This method of adjusting GLP-1 agonist therapy based on patient-specific parameters allows for a personalized approach to treatment, potentially improving efficacy and patient compliance.

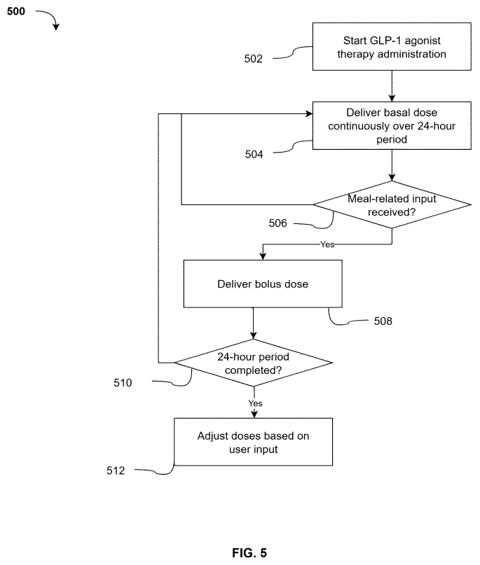

FIG. 5 illustrates a flowchart for a method of administering GLP-1 agonist therapy with multiple bolus doses using the drug delivery system 100. The method begins with a step 502 of initiating GLP-1 agonist therapy administration.

Following initiation, the method proceeds to a step 504 of delivering a basal dose of the GLP-1 agonist continuously over a 24-hour period using the pump 104. The basal dose module 202 of the controller 200 may manage this continuous delivery.

The method then moves to a decision point 506 where a determination is made whether a meal-related input has been received. In some cases, the user interface 108 may be configured to receive meal-related inputs from a patient or healthcare provider.

If a meal-related input is received, the method follows the “Yes” branch to a step 508 where a bolus dose of the GLP-1 agonist is delivered. The bolus dose module 204 of the controller 200 may manage the delivery of this bolus dose. In some cases, multiple bolus doses may be delivered at different times within the 24-hour period, each in response to a separate meal-related input.

If no meal-related input is received, or after delivering a bolus dose, the method proceeds to a decision point 510 where a determination is made whether the 24-hour period is completed.

If the 24-hour period is not completed, the method follows the “No” branch and returns to step 504 to continue delivering the basal dose. This loop allows for the delivery of multiple bolus doses at different times within the 24-hour period, as needed based on meal-related inputs.

If the 24-hour period is completed, the method follows the “Yes” branch to a step 512 where doses are adjusted based on user input. The dose adjustment module 206 of the controller 200 may perform these adjustments. In some cases, the user interface 108 may be used to receive user input for adjusting the basal dose and the bolus doses.

After adjusting the doses, the method returns to step 504 to begin delivering the adjusted basal dose for the next 24-hour period. This cyclical process allows for ongoing administration and adjustment of the GLP-1 agonist therapy based on meal-related inputs and user feedback.

In some cases, the glucose sensor 110 may provide glucose readings to the glucose reading module 210 of the controller 200. These readings may be used by the dose adjustment module 206 to further refine the basal and bolus doses during the adjustment step 512.

The patient parameters module 208 of the controller 200 may also provide relevant patient-specific information, such as body weight or meal size data, to inform the dose adjustments in step 512.

This method of administering GLP-1 agonist therapy with multiple bolus doses allows for a flexible and personalized approach to treatment, potentially improving efficacy and patient compliance by adapting to individual meal patterns and metabolic needs.

Overall, the drug delivery system 100 and associated methods described herein may provide several improvements over conventional GLP-1 agonist therapies. Traditional GLP-1 agonist treatments typically involve fixed weekly injections that may not account for individual patient variability or daily metabolic fluctuations. In contrast, the present system may offer a more personalized and flexible approach to GLP-1 agonist administration.

The basal-bolus delivery approach may allow for continuous background medication delivery while providing additional doses in response to specific physiological needs, such as meals. This dual-component system may help maintain more consistent therapeutic levels throughout the day compared to single weekly injections. The controller 200 may enable independent adjustment of basal and bolus components, allowing healthcare providers to tailor treatment to individual patient requirements.

The system may also provide improved patient convenience through programmable dosing regimens that can be adjusted based on real-time glucose readings from the glucose sensor 110 and patient-specific parameters stored in the patient parameters module 208. This responsive approach may help optimize therapeutic outcomes while potentially reducing side effects through more precise dose control.

Additionally, the drug delivery system 100 may offer enhanced flexibility in treatment initiation and modification. Patients may begin with basal dosing alone and gradually add bolus components as tolerated, or adjust their regimen based on changing lifestyle factors or clinical needs. The system may also allow for temporary dose reductions or treatment holidays if gastrointestinal side effects occur, potentially improving long-term treatment adherence compared to fixed dosing schedules.

For the embodiments and methods described herein, dosing may be expressed as a multiple of total daily dosing exposure for the given GLP1(+) therapy (illustrative example: exenatide) where total daily exposure can be in the range of 20-200% of total daily recommended dose by US FDA labelling. The balance of basal to bolus dosing may be expressed as a balance of total basal dose for the 24 hours and the total composite of bolus doses during the 24 hours, independent of the actual total daily dose. The range for the proportion of basal dosing can be 10-90% for the given 24 hours with the total composite bolus dosing making up the remainder of this proportion. The number of discrete bolus doses, independent of basal dosing, may, for example, number one to ten with the ability to vary doses at each time point across a given 24 hours. Total daily dose, dose balance between basal and net bolus, and bolus dosing at discrete time points may vary over time per clinical need. Up-titration of dosing at initiation may take the form of (i) introduction of basal dosing at the outset with addition of bolus dosing at a later time point, (ii) introduction of bolus dosing with one or more discrete boluses doses at the outset with later introduction of basal dosing, or (iii) the introduction of a basal-bolus dosing regimen at the outset with flexibility to up-titrate either or both basal and bolus dosing components as required. As contemplated herein, reduction or cessation of treatment may take the form of (i) complete cessation, (ii) halting or reducing one, more or all bolus doses first, (iii) halting or reducing basal dosing first, or (iv) reducing both basal and bolus components of therapy in tandem.

A number of implementations have been described. Nevertheless, it will be understood that various modifications may be made without departing from the spirit and scope of the disclosure. Accordingly, other implementations are within the scope of the following claims.

1. A drug delivery system for administering a GLP-1 agonist therapy, comprising:
a reservoir configured to contain a GLP-1 agonist;
a pump in fluid communication with the reservoir and configured to deliver a GLP-1 agonist;
a controller operatively coupled to the pump; and
a user interface operatively coupled to the controller,
wherein the controller is programmed to:
deliver a basal dose of the GLP-1 agonist continuously over a 24-hour period; and
deliver at least one bolus dose of the GLP-1 agonist in addition to the basal dose.

2. The drug delivery system of claim 1, wherein the basal dose and the at least one bolus dose are independently adjustable.

3. The drug delivery system of claim 1, wherein the controller is further programmed to adjust the basal dose and the at least one bolus dose based on patient-specific parameters.

4. The drug delivery system of claim 3, wherein the patient-specific parameters include at least one of body weight, meal size, time of day, or glucose levels.

5. The drug delivery system of claim 1, wherein the controller is programmed to deliver multiple bolus doses at different times within a 24-hour period.

6. The drug delivery system of claim 1, wherein the basal dose comprises 10% to 90% of a total daily dose of the GLP-1 agonist.

7. The drug delivery system of claim 1, further comprising a reservoir containing the GLP-1 agonist, wherein the GLP-1 agonist is a short-acting GLP-1 receptor agonist.

8. The drug delivery system of claim 1, wherein the user interface is configured to receive user input for adjusting the basal dose and the at least one bolus dose.

9. The drug delivery system of claim 1, further comprising a glucose sensor operatively coupled to the controller, wherein the controller is programmed to adjust the basal dose and the at least one bolus dose based on glucose readings from the glucose sensor.

10. The drug delivery system of claim 1, wherein the controller is programmed to deliver the at least one bolus dose in response to a meal-related input received through the user interface.

11. A method of administering a GLP-1 agonist therapy, comprising:
delivering a basal dose of a GLP-1 agonist continuously over a 24-hour period using a pump; and
delivering at least one bolus dose of the GLP-1 agonist in addition to the basal dose using the pump.

12. The method of claim 11, further comprising:
independently adjusting the basal dose and the at least one bolus dose.

13. The method of claim 11, further comprising adjusting the basal dose and the at least one bolus dose based on patient-specific parameters.

14. The method of claim 13, wherein the patient-specific parameters include at least one of body weight, meal size, time of day, or glucose levels.

15. The method of claim 11, wherein delivering the at least one bolus dose comprises delivering multiple bolus doses at different times within the 24-hour period.

16. The method of claim 11, wherein the basal dose comprises 10% to 90% of a total daily dose of the GLP-1 agonist.

17. The method of claim 11, wherein the GLP-1 agonist is a short-acting GLP-1 receptor agonist.

18. The method of claim 11, further comprising receiving user input through a user interface for adjusting the basal dose and the at least one bolus dose.

19. The method of claim 11, further comprising:
adjusting the basal dose and the at least one bolus dose based on glucose readings from a glucose sensor operatively coupled to the pump.

20. The method of claim 11, wherein delivering the at least one bolus dose comprises delivering the bolus dose in response to a meal-related input received through a user interface.
